(19) United States
(12) Patent Application Publication
(10) Pub. No.: US 2022/0008689 A1
(43) Pub. Date: Jan. 13, 2022

(54) SLEEP-AID DEVICE AND METHOD THEREOF

(71) Applicant: AWNT LIMITED, New Taipei (TW)

(72) Inventors: Yi-Chang Liu, New Taipei (TW); Li-Min Sun, Beijing (CN)

(21) Appl. No.: 17/368,894

(22) Filed: Jul. 7, 2021

(30) Foreign Application Priority Data
Jul. 7, 2020 (TW) 109122922

(51) Int. Cl. A61M 21/02

(52) U.S. Cl. CPC A61M 21/02; A61M 2210/0662; A61M 2205/3303

(57) ABSTRACT

The present disclosure relates a sleep-aid device which includes a brainwave sensor, a processor, and an output device disposed in a case. The brainwave sensing module senses a brainwave by contacting an ear and generates a brainwave reference signal. The processor processes the brainwave reference signal when received the brainwave reference signal, then amplifies at least one of the sound feature point audios conforming to the brainwave data to generate a brainwave sound signal with a brainwave frequency, further suppressing or shielding the sound feature point audios of the other sound frequencies not being amplified to generate adjusted sound signals. The processor compares the brainwave frequency by a mental parameter and generates a sober signal or a sleep signal according to a mental comparison result, providing the output device determines whether outputting a sleep media with a sleep frequency according to the sober signal or the sleep signal.

### CROSS-REFERENCE TO RELATED APPLICATION

The present disclosure is based upon and claims priority to Taiwan Patent Application No. 109122922, filed on Jul. 7, 2020, the content of which is incorporated herein by reference in its entirety for all purposes.

### BACKGROUND

### 1. Technical Field

The present disclosure relates to a field of sleep-aid, and more particularly to a sleep-aid device and method thereof of determining whether to trigger the related sleep-aid operation based on the mental status of a user.

### 2. Description of the Related Art

According to statistics, approximately a quarter of the world's population are suffered from sleep problems. People usually take sleeping pills to help them sleep at night, which causes the global usage of sleeping pills to skyrocket to about 880 million. Although it has a rapid effect by using sleeping pills to improve sleep quality, the result of long-term use will cause a great adverse reaction to affect the health of the human body and may also cause drug addiction, furthermore, it will be harder to sleep without the sleeping pills. Thus, a number of medical experts are elaborately researching how to improve sleep quality in order to solve the above-mentioned problems.

In the prior art, casting sleep music or related sound frequencies is invented to help people sleep, but it has to be operated by a user to play or turn it off, which cannot be determined based on, for example, the mental status of the user (that is, determining whether fall asleep). Therefore, it is not only inconvenient in operation, but also wasting great power and shortening the service life of the device (e.g. depreciation rate) since the related devices that playing the sleep music will not be shut down correspondingly if the user is asleep.

Therefore, how to provide a technology that can determine whether outputting related sleep music based on the mental status of the user is required to solve the problems existing in the prior art.

### SUMMARY

The purpose of the present disclosure is to provide a sleep-aid device that primarily utilizes a processor to process a brainwave reference signal after a brainwave sensor sensing a brainwave to determine a mental status of a user, and an output device determines whether outputting a sleep media with a sleep frequency based on the mental status of the user, effectively solving the problems of the prior art thereby.

Based on the purpose of the present disclosure, a sleep-aid device is provided, which comprises: a case arranged on an ear of a user; a brainwave sensor disposed in the case to sense a brainwave by contacting the ear, and generating a brainwave reference signal based on a sensing result; a processor disposed in the case and connected to the brainwave sensor, the processor receiving the brainwave reference signal and extracting sound frequencies from the brainwave reference signal, comparing sound feature points in the sound frequencies with a plurality of frequency data, extracting sound feature point audios of the sound frequency in the sound feature points conforming to a brainwave data by using a deep learning algorithm, amplifying at least one of the sound feature point audios conforming to the brainwave data to generate a brainwave sound signal with a brainwave frequency, and suppressing or shielding the sound feature point audios of the other sound frequencies not being amplified to generate adjusted sound signals, wherein the processor compares the brainwave sound signal with the brainwave frequency based on at least one mental parameter to generate a sober signal or a sleep signal according to a mental comparison result; and an output device disposed in the case and connected to the processor, the output device storing at least one sleep media with a sleep frequency, wherein the at least one sleep media with the sleep frequency is outputted by the output device when the output device receives the sober signal, or the output device is in a standby state when the output device receives the sleep signal.

In an embodiment, the brainwave sensor includes: a sensing electrode sensing the brainwave by contacting the ear to generate a sensed brainwave, the brainwave sensor connected to another brainwave sensor to receive a reference brainwave generated by a brainwave sensed by a sensing electrode of the another brainwave sensor; wherein the sensing brainwave and the reference brainwave are calculated by the brainwave sensor to generate the brainwave reference signal.

In an embodiment, the brainwave sensor includes: a first sensing electrode sensing the brainwave by contacting an upper part of the ear to generate a sensing brainwave; a second sensing electrode sensing the brainwave by contacting a lower part of the ear to generate a reference brainwave; wherein the sensing brainwave and the reference brainwave are calculated by the brainwave sensor to generate the brainwave reference signal.

In an embodiment, the brainwave sensor performs the action of sensing the brainwave in a sensing period interval.

In an embodiment, the sleep-aid device further includes: a register connected to the processor and the output device for storing the brainwave reference signal, the mental parameter, the sober signal, the sleep signal, the sleep media, or any combinations of two or more thereof.

In an embodiment, the sleep-aid device further includes: a configuration module stored a configuration data, the configuration module connected to the brainwave sensor to transmit the configuration data, and a daily operation period determined based on the configuration data by the brainwave sensor.

In an embodiment, the sleep-aid device further includes: a parameter adjuster connected to the processor to receive the brainwave reference signal, wherein the parameter adjuster compares the brainwave reference signal based on an external sleep configuration data when the external sleep configuration data is received, and adjusts the mental parameter according to a comparison result.

In an embodiment, a parameter adjusting interface is provided by the parameter adjuster to receive the external sleep configuration data, or the parameter adjuster connected to a sleep sensor to receive the external sleep configuration data generated by the sleep sensor.

Another purpose of the present disclosure is to provide a sleep-aid method that that primarily utilizes a processor to process a brainwave reference signal after a brainwave sensor sensing a brainwave to determine a mental status of a user, and an output device determines whether outputting a sleep media with a sleep frequency based on the mental status of the user, effectively solving the problems of the prior art thereby.

Based on another purpose of the present disclosure, a sleep-aid method is provided, which applicable to a sleep-aid device, the sleep-aid method comprises: sensing a brainwave by contacting an ear of a user by a brainwave sensor; generating a brainwave reference signal based on a sensing result by the brainwave sensor; receiving the brainwave reference signal and extracting sound frequencies from the brainwave reference signal by a processor; comparing sound feature points in the sound frequencies with a plurality of frequency data by the processor; extracting sound feature point audios of the sound frequency in the sound feature points conforming to a brainwave data by using a deep learning algorithm; amplifying at least one of the sound feature point audios conforming to the brainwave data by the processor to generate a brainwave sound signal with a brainwave frequency; suppressing or shielding the sound feature point audios of the other sound frequencies not being amplified by the processor to generate adjusted sound signals; comparing the brainwave sound signal with the brainwave frequency based on at least one mental parameter by the processor to generate a sober signal or a sleep signal; outputting at least one sleep media with a sleep frequency by the output device when the sober signal received by the output device; being in a standby state by the output device when the sleep signal received by the output device.

In an embodiment, the sleep-aid method further includes: sensing the brainwave by contacting the ear by a sensing electrode of the brainwave sensor to generate a sensed brainwave; receiving a reference brainwave generated by a brainwave sensed by a sensing electrode of another brainwave sensor by the brainwave sensor; calculating the sensing brainwave and the reference brainwave by the brainwave sensor to generate the brainwave reference signal.

In an embodiment, the sleep-aid method further includes: sensing the brainwave by contacting an upper part of the ear by a first sensing electrode of the brainwave sensor to generate a sensing brainwave; sensing the brainwave by contacting a lower part of the ear by a second sensing electrode of the brainwave sensor to generate a reference brainwave; calculating the sensing brainwave and the reference brainwave by the brainwave sensor to generate the brainwave reference signal.

In an embodiment, the brainwave sensor performs the action of sensing the brainwave in a sensing period interval.

In an embodiment, the sleep-aid method further includes: storing the brainwave reference signal, the mental parameter, the sober signal, the sleep signal, the sleep media with the sleep frequency, or any combinations of two or more thereof by a register.

In an embodiment, the sleep-aid method further includes: transmitting the configuration data to the brainwave sensor by a configuration module; determining a daily operation period based on the configuration data by the brainwave sensor.

In an embodiment, the sleep-aid method further includes: comparing the brainwave reference signal based on an external sleep configuration data when the external sleep configuration data received by a parameter adjuster; adjusting the mental parameter according to a comparison result by the parameter adjuster.

In an embodiment, a parameter adjusting interface is provided by the parameter adjuster to receive the external sleep configuration data, or the parameter adjuster connected to a sleep sensor to receive the external sleep configuration data generated by the sleep sensor.

In order to lead the purposes, features, and advantages of the present disclosure as described above can be obviously understandable, the specific embodiments listed in the drawings are described in detail below.

### BRIEF DESCRIPTION OF THE DRAWINGS

FIG. 1 illustrates a schematic diagram of elements configuration of the present disclosure.

FIG. 2 illustrates an embodiment of a schematic diagram of another brainwave sensor configuration of the present disclosure.

FIG. 3 illustrates an embodiment of a schematic diagram of brainwave sensor configuration of the present disclosure.

FIG. 4 illustrates a schematic diagram of an configuration module configuration of the present disclosure.

FIG. 5 illustrates a schematic diagram of an parameter adjuster configuration of the present disclosure.

FIG. 6 illustrates a diagram of steps process of the present disclosure.

### DESCRIPTION OF THE PREFERRED EMBODIMENTS

The advantages, features, and technical methods of the present disclosure are to be explained in detail with reference to the exemplary embodiments and the drawings for a better understanding of the present disclosure. Moreover, the present disclosure may be realized in different forms, and should not be construed as being limited to the embodiments set forth herein. Conversely, for a person of ordinary skill in the art, the embodiments provided shall make the present disclosure convey the scope more thoroughly, comprehensively, and completely. In addition, the present disclosure shall be defined only by the appended claims.

The terms used in the present disclosure are only for the purpose of describing specific embodiments, not intended to limit the present disclosure. Unless otherwise defined, the technical terms or scientific terms used in the present disclosure shall have the usual meanings understood by those with ordinary skills in the field to which this disclosure belongs. The “one” or “a” or other similar words used in the specification of the present disclosure and the claims do not mean a limit of quantity, but mean that there is at least one. Unless otherwise stated, “including” or “comprising” or other similar words mean that the elements or objects before “including” or “comprising” contains the elements or objects or their equivalents listed after “including” or “comprising”, and other elements or objects are not excluded. Similar words such as “connection” or “connect” are not limited to physical or mechanical connections, and may include electrical connections, no matter whether direct or indirect. The singular forms of “a”, “the” and “this” used in the specification and claims of this disclosure are also intended to include plural forms, unless the context clearly indicates other meanings. It should also be understood that the term “and/or” as used herein refers to any or all possible combinations of one or more associated listed items.

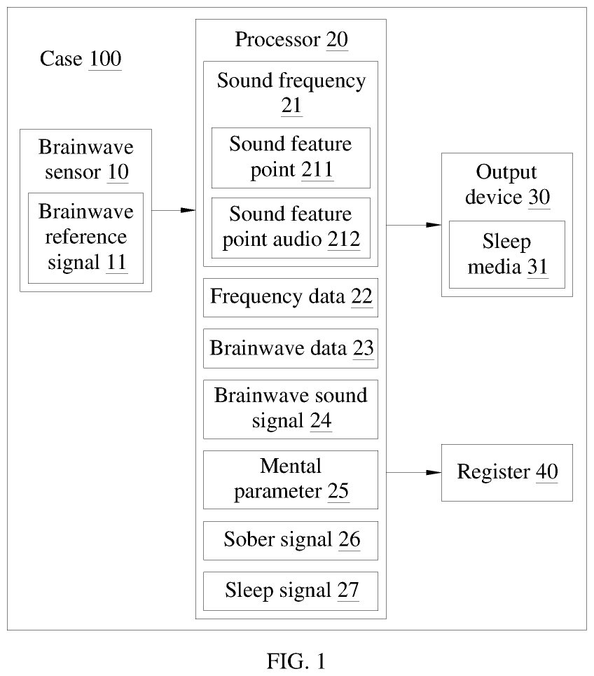

Please refer to FIG. 1 which is a schematic diagram of elements configuration of the present disclosure. As shown in the figure, the present disclosure primarily consists of a brainwave sensor 10, a processor 20, and an output device 30 disposed in a case 100, and the case 100 is arranged on an ear of a user. The brainwave sensor 10 may be a related device that configured to sense a brainwave of a user. In an embodiment, the brainwave sensor 10 arranged a contact point that contacting with the ear to sense the brainwave of the user by the contact point. Furthermore, a brainwave reference signal 11 can be transformingly generated by the brainwave sensor 10 based on a brainwave variation (that is, the sensing result) sensed by the contact point.

The processor 20 may specifically be a central processing unit or other devices capable of data processing. The processor 20 will extract sound frequencies 21 (e.g. brainwave and other noises) from the brainwave reference signal 11 when the processor 20 receives the brainwave reference signal 11 by connecting to the brainwave sensor 10. In order to effectively distinguish the differences between the sound frequencies 21 and compare a brainwave of a user with the differences, so that the processor 20 will compare sound feature points 211 in the at least one sound frequency 21 with a plurality of frequency data 22 after each of the sound frequencies 21 is extracted.

Sound feature point audios 212 of the sound frequency 21 in the sound feature points 211 conforming to the brainwave data 23 (e.g. the brainwave data 23 is stored in the processor 20) will be extracted by using a deep learning algorithm by the processor 20, and at least one of the sound feature point audios 212 conforming to the brainwave data 23 (e.g. the brainwave data 23 records a brainwave frequency of human) is amplified by the processor 20 to generate a brainwave sound signal 24 with the brainwave frequency, and the sound feature point audios 212 in the other sound frequencies 21 those are not amplified can be suppressed or shielded to generate adjusted sound signals. Consequently, the brainwave sound signal 24 with the brainwave frequency of the user can be highlighted by the afore-mentioned noise-canceling process (that is, amplifying the brainwave sound signal 24 with the brainwave frequency and suppressing or shielding the sound feature point audios 212 of the other sound frequencies 21), to facilitate subsequent determination of the processor 20.

Generally speaking, when a person is in a state of thinking, reasoning, stressful, intensive, uncomfortable, or anxious, the brainwave of the person will be a β wave (e.g. 12 to 38 Hz), but if the person is in a state of distracting, relaxing, drifting, spacing out, absent-minded, and closing the eyes, the brainwave of the person will be a wave (e.g. 8 to 12 Hz). Moreover, the brainwave can be low-frequency θ wave (4 to 8 Hz) and δ wave (0.5 to 4 Hz) when the person closed his eyes and fell asleep. Therefore, the processor 20 may compare the brainwave sound signal 24 with the brainwave frequency based on at least one mental parameter 25 (e.g. the above information) which stored in the processor 20 after the noise-canceling process is completed by the processor 20, and generate a sober signal 26 or a sleep signal 27 according to a mental comparison result.

After that, the output device 30 can receive the sober signal 26 or the sleep signal 27 by connecting with the processor 20, wherein the sober signal 26 represents the user is in a state of sobering, so that the output device 30 outputs at least one sleep media 31 with a sleep frequency which stored in the output device 30 when the sober signal 26 is received by the output device 30, to help the user sleep. The sleep signal 27 represents the user is in a state of sleeping, so that the output device 30 is in a standby state when the output device 30 receives the sleep signal 27. In addition, the brainwave sensor 10 may perform the action of sensing the brainwave in a sensing period (e.g. 5 minutes or 10 minutes) interval, to perform another action of sensing the brainwave for continuously sensing the state of the user and avoiding the sleep-aid device of the present disclosure is still playing the sleep media 31 after the user falls asleep, in order to effectively sense the state of the user during sleep.

Furthermore, a register 40 can be included in the sleep-aid device of the present disclosure, to store the brainwave reference signal 11, the mental parameter 25, the sober signal 26, the sleep signal 27, the sleep media 31, or any combinations of two or more thereof by connecting to the processor 20 and the output device 30.

Moreover, since the conversion or performing between digital signals or analog signals is a prior art, in the above-mentioned signal receiving or outputting actions, the actions known in the prior art will not be repeated.

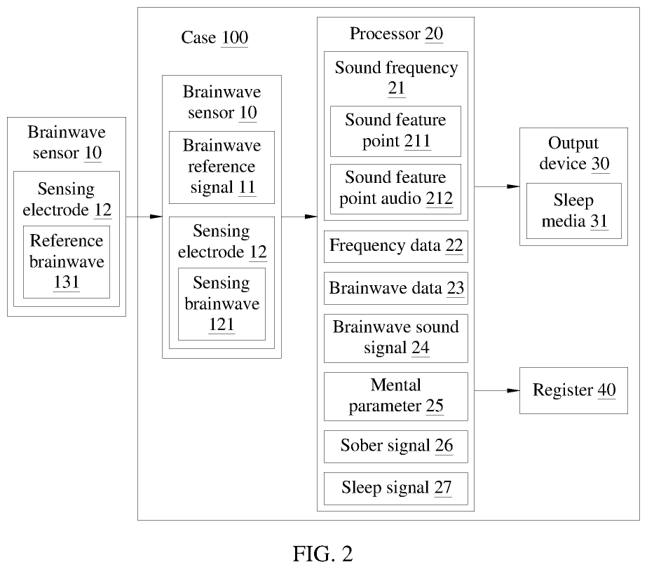

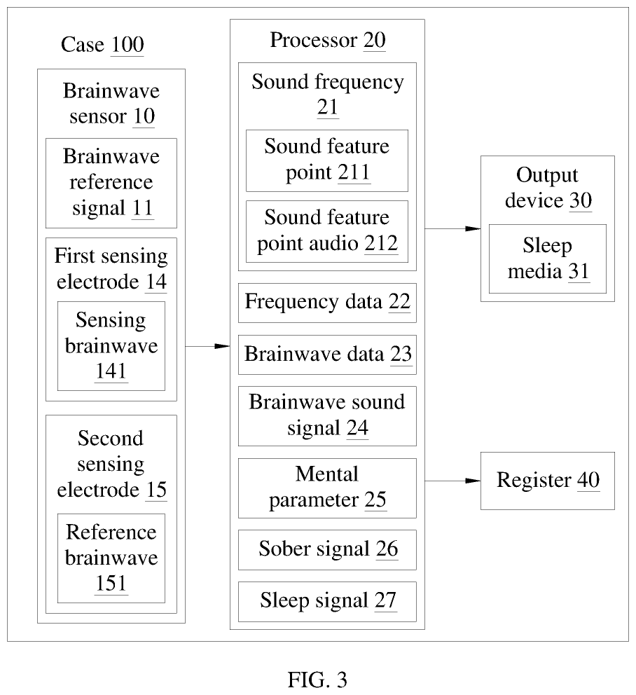

Please further refer to FIGS. 2 and 3, which is an embodiment of a schematic diagram of another brainwave sensor configuration and an embodiment of a schematic diagram of brainwave sensor configuration of the present disclosure. As shown in the figures, besides, using one contact point to sense the brainwave, two or more of the contact points can be also provided by the brainwave sensor 10 of the present disclosure, to sense the brainwave of the user. For example, as shown in the FIG. 2, a sensing electrode 12 is included by the brainwave sensor 10 to sense the brainwave by contacting the ear to generate a sensed brainwave 121, furthermore, another brainwave sensor 10 can be connected to the brainwave sensor 10 for receiving a reference brainwave 131 generated by a brainwave sensed by a sensing electrode 12 of the another brainwave sensor 10, then calculating the sensing brainwave 121 and the reference brainwave 131 by the brainwave sensor 10 to generate the brainwave reference signal 11.

As shown in the FIG. 3, a first sensing electrode 14 and a second sensing electrode 15 can be included by the brainwave sensor 10, for sensing the brainwave by contacting an upper part of the ear by the first sensing electrode 14 to generate a sensing brainwave 141, and sensing the brainwave by contacting a lower part of the ear by the second sensing electrode 15 to generate a reference brainwave 151. Accordingly, the sensing brainwave 121 and the reference brainwave 131 can be calculated by the brainwave sensor 10 to generate the brainwave reference signal 11 after two sensed data are sensed by two contact points (e.g. the first sensing electrode 14 and the second sensing electrode 15).

Thus, the brainwave reference signal 11 of the present disclosure can be generated whether by one or two contact points, to performing subsequent determination of the mental parameter 25.

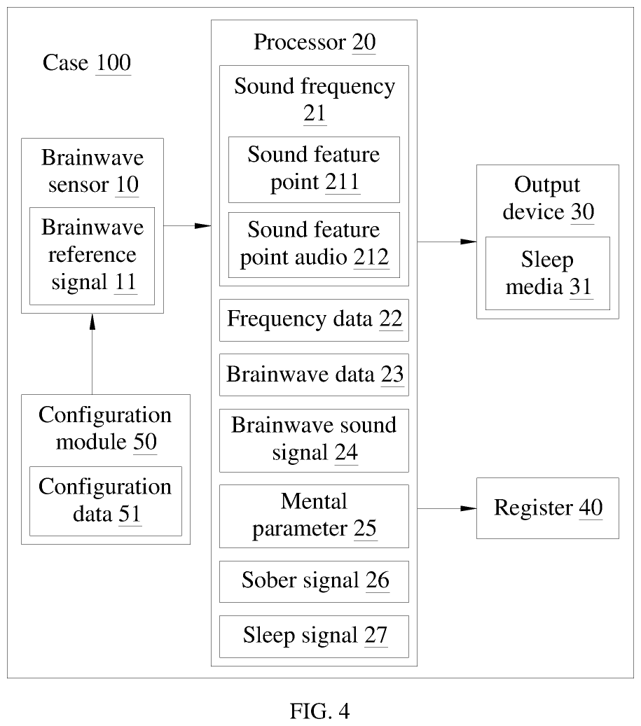

Please further refer to FIG. 4, which is a schematic diagram of an configuration module configuration of the present disclosure. As shown in the figure, in order to provide the determination of the brainwave sensor 10 can be operated when the user falls asleep or going sleep in a state of the user during sleep. A configuration module 50 can be included by the sleep-aid device of the present disclosure, the configuration module 50 stores a configuration data 51 and disposed in the case 100, the configuration data 51 may be transmitted by the configuration module 50 when the brainwave sensor 10 is connected to the configuration module 50, to allow the brainwave sensor 10 determining a sleep period of the user with the configuration data 51, and determining a daily operation period of the brainwave sensor 10 based on the sleep period of the user.

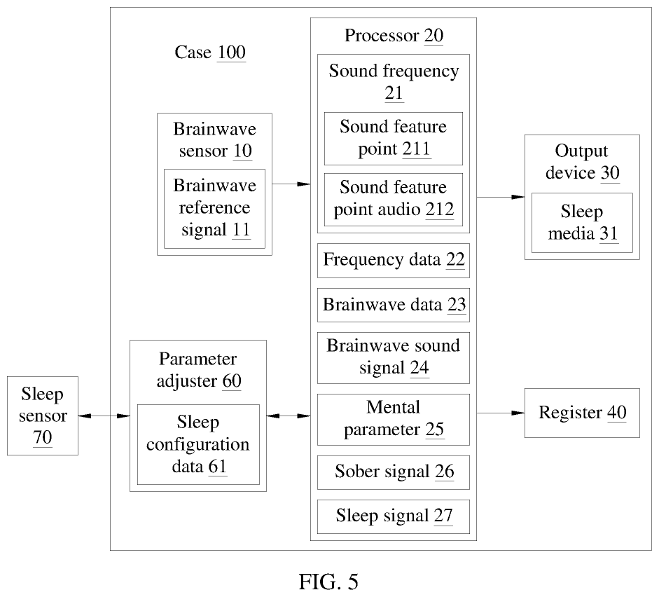

Please further refer to FIG. 5, which is a schematic diagram of an parameter adjuster configuration of the present disclosure. As shown in the figure, in order to provide accurately determination of a mental condition of the user, the mental parameter 25 can be adjusted by the condition of the user. A parameter adjuster 60 can be further included by the sleep-aid device of the present disclosure, the parameter adjuster 60 receives the brainwave reference signal 11 by connecting to the processor 20, compares the brainwave reference signal 11 based on an external sleep configuration data 61 when the external sleep configuration data 61 is received (e.g. a parameter adjusting interface is provided by the parameter adjuster 60 to receive the external sleep configuration data 61, or the parameter adjuster 60 is connected to a sleep sensor 70 to receive the external sleep configuration data 61 generated by the sleep sensor 70) by the parameter adjuster 60, then adjusts the mental parameter 25 according to a comparison result accordingly.

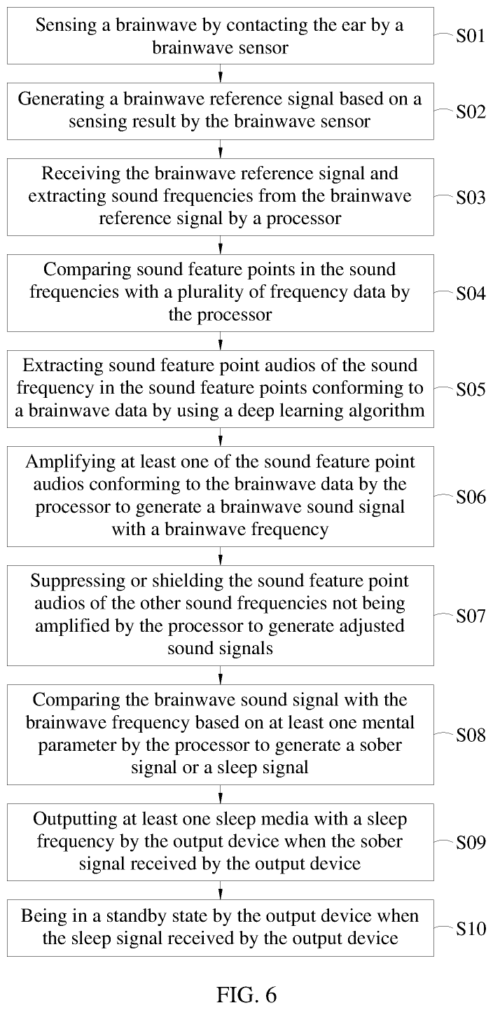

Please further refer to FIG. 6, which is a diagram of steps process of the present disclosure. As shown in the figure, the effect of determining whether trigging the related sleep-aid operation based on the mental status of the user as the above-mentioned can be achieved by the following steps process, which includes:

S01: sensing a brainwave by contacting the ear by a brainwave sensor;

S02: generating a brainwave reference signal based on a sensing result by the brainwave sensor;

S03: receiving the brainwave reference signal and extracting sound frequencies from the brainwave reference signal by a processor;

S04: comparing sound feature points in the sound frequencies with a plurality of frequency data by the processor;

S05: extracting sound feature point audios of the sound frequency in the sound feature points conforming to a brainwave data by using a deep learning algorithm;

S06: amplifying at least one of the sound feature point audios conforming to the brainwave data by the processor to generate a brainwave sound signal with a brainwave frequency;

S07: suppressing or shielding the sound feature point audios of the other sound frequencies not being amplified by the processor to generate adjusted sound signals;

S08: comparing the brainwave sound signal with the brainwave frequency based on at least one mental parameter by the processor to generate a sober signal or a sleep signal;

S09: outputting at least one sleep media with a sleep frequency by the output device when the sober signal received by the output device;

S10: being in a standby state by the output device when the sleep signal received by the output device.

Accordingly, the present disclosure can determine whether trigging the related sleep-aid operation based on the mental status of the user to facilitate the user using the sleep-aid device of the present disclosure thereby.

The above description is merely illustrative rather than restrictive. Any equivalent modifications or alterations without departing from the spirit and scope of the present disclosure are intended to be included in the following claims.

In summary, regardless of the purposes, means, and effects of the present disclosure, which is showing the technical characteristics that are different from the prior art, and it is invented suitable for practical use, and also in compliance with the patent requirements of the present disclosure. Praying that the patent will be granted as soon as possible, so as to benefit society.

What is claimed is:

1. A sleep-aid device, comprising:
a case arranged on an ear of a user;
a brainwave sensor disposed in the case to sense a brainwave by contacting the ear, and generating a brainwave reference signal based on a sensing result;
a processor disposed in the case and connected to the brainwave sensor, the processor receiving the brainwave reference signal and extracting sound frequencies from the brainwave reference signal, comparing sound feature points in the sound frequencies with a plurality of frequency data, extracting sound feature point audios of the sound frequency in the sound feature points conforming to a brainwave data by using a deep learning algorithm, amplifying at least one of the sound feature point audios conforming to the brainwave data to generate a brainwave sound signal with a brainwave frequency, and suppressing or shielding the sound feature point audios of the other sound frequencies not being amplified to generate adjusted sound signals, wherein the processor compares the brainwave sound signal with the brainwave frequency based on at least one mental parameter to generate a sober signal or a sleep signal according to a mental comparison result; and
an output device disposed in the case and connected to the processor, the output device storing at least one sleep media with a sleep frequency, wherein the at least one sleep media with the sleep frequency is outputted by the output device when the output device receives the sober signal, or the output device is in a standby state when the output device receives the sleep signal.

2. The sleep-aid device according to claim 1, wherein the brainwave sensor includes:
a sensing electrode sensing the brainwave by contacting the ear to generate a sensed brainwave, the brainwave sensor connected to another brainwave sensor to receive a reference brainwave generated by a brainwave sensed by a sensing electrode of the another brainwave sensor;
wherein the sensing brainwave and the reference brainwave are calculated by the brainwave sensor to generate the brainwave reference signal.

3. The sleep-aid device according to claim 1, wherein the brainwave sensor includes:
a first sensing electrode sensing the brainwave by contacting an upper part of the ear to generate a sensing brainwave;
a second sensing electrode sensing the brainwave by contacting a lower part of the ear to generate a reference brainwave;
wherein the sensing brainwave and the reference brainwave are calculated by the brainwave sensor to generate the brainwave reference signal.

4. The sleep-aid device according to claim 1, wherein the brainwave sensor performs the action of sensing the brainwave in a sensing period interval.

5. The sleep-aid device according to claim 1, further comprising:
a register connected to the processor and the output device for storing the brainwave reference signal, the mental parameter, the sober signal, the sleep signal, the sleep media, or any combinations of two or more thereof.

6. The sleep-aid device according to claim 1, further comprising:
a configuration module storing a configuration data, the configuration module connected to the brainwave sensor to transmit the configuration data, and a daily operation period determined based on the configuration data by the brainwave sensor.

7. The sleep-aid device according to claim 1, further comprising:
a parameter adjuster connected to the processor to receive the brainwave reference signal, wherein the parameter adjuster compares the brainwave reference signal based on an external sleep configuration data when the external sleep configuration data is received, and adjusts the mental parameter according to a comparison result.

8. The sleep-aid device according to claim 7, wherein a parameter adjusting interface is provided by the parameter adjuster to receive the external sleep configuration data, or the parameter adjuster connected to a sleep sensor to receive the external sleep configuration data generated by the sleep sensor.

9. A sleep-aid method, applicable to a sleep-aid device, the sleep-aid method comprising:
sensing a brainwave by contacting an ear of a user by a brainwave sensor;
generating a brainwave reference signal based on a sensing result by the brainwave sensor;
receiving the brainwave reference signal and extracting sound frequencies from the brainwave reference signal by a processor;
comparing sound feature points in the sound frequencies with a plurality of frequency data by the processor;
extracting sound feature point audios of the sound frequency in the sound feature points conforming to a brainwave data by using a deep learning algorithm;
amplifying at least one of the sound feature point audios conforming to the brainwave data by the processor to generate a brainwave sound signal with a brainwave frequency;
suppressing or shielding the sound feature point audios of the other sound frequencies not being amplified by the processor to generate adjusted sound signals;
comparing the brainwave sound signal with the brainwave frequency based on at least one mental parameter by the processor to generate a sober signal or a sleep signal;
outputting at least one sleep media with a sleep frequency by the output device when the sober signal received by the output device;
being in a standby state by the output device when the sleep signal received by the output device.

10. The sleep-aid method according to claim 9, further comprising:
sensing the brainwave by contacting the ear by a sensing electrode of the brainwave sensor to generate a sensed brainwave;
receiving a reference brainwave generated by a brainwave sensed by a sensing electrode of another brainwave sensor by the brainwave sensor;
calculating the sensing brainwave and the reference brainwave by the brainwave sensor to generate the brainwave reference signal.

11. The sleep-aid method according to claim 9, further comprising:
sensing the brainwave by contacting an upper part of the ear by a first sensing electrode of the brainwave sensor to generate a sensing brainwave;
sensing the brainwave by contacting a lower part of the ear by a second sensing electrode of the brainwave sensor to generate a reference brainwave;
calculating the sensing brainwave and the reference brainwave by the brainwave sensor to generate the brainwave reference signal.

12. The sleep-aid method according to claim 9, wherein the brainwave sensor performs the action of sensing the brainwave in a sensing period interval.

13. The sleep-aid method according to claim 9, further comprising:
storing the brainwave reference signal, the mental parameter, the sober signal, the sleep signal, the sleep media, or any combinations of two or more thereof by a register.

14. The sleep-aid method according to claim 9, comprising:
transmitting the configuration data to the brainwave sensor by a configuration module;
determining a daily operation period based on the configuration data by the brainwave sensor.

15. The sleep-aid method according to claim 9, further comprising:
comparing the brainwave reference signal based on an external sleep configuration data when the external sleep configuration data received by a parameter adjuster;
adjusting the mental parameter according to a comparison result by the parameter adjuster.

16. The sleep-aid method according to claim 15, wherein a parameter adjusting interface is provided by the parameter adjuster to receive the external sleep configuration data, or the parameter adjuster connected to a sleep sensor to receive the external sleep configuration data generated by the sleep sensor.
